(19) United States
(12) Patent Application Publication
(10) Pub. No.: US 2008/0312745 A1
(43) Pub. Date: Dec. 18, 2008

(54) ENDOPROSTHESIS WITH CONVEX CONFIGURATION

(75) Inventors: Arnold Keller, Kayhude (DE); Hakon Kofoed, Charlottenlund (DK)

Correspondence Address: MORRISON & FOERSTER LLP, 1650 TYSONS BOULEVARD, SUITE 400, MCLEAN, VA, 22102, US

(73) Assignee: Waldemar Link GmbH & Co. KG, Hamburg (DE)

(21) Appl. No.: 12/138,101

(22) Filed: Jun. 12, 2008

Related U.S. Application Data
(60) Provisional application No. 60/929,080, filed on Jun. 12, 2007.

(51) Int. Cl. A61F 2/30

(52) U.S. Cl. 623/18.11

(57) ABSTRACT

The invention relates to an endoprosthesis for replacement of a joint, in which slide surfaces that have different contours and correspondingly define movement planes for bearing are formed by an intermediate part. According to the invention, the intermediate part has a projection, preferably a convex projection, on at least one side face, in order to avoid fibrosis.

This application claims priority to U.S. Provisional Application No. 60/929,080 filed Jun. 12, 2007, the contents of which are incorporated herein in their entirety.

### FIELD OF THE INVENTION

The invention relates to an endoprosthesis for replacement of a joint, comprising a component which is to be connected to a lower bone and which has a top slide surface, a component which is to be connected to an upper bone and which has a bottom slide surface, and an intermediate part which, on its bottom and top, has in each case a mating slide surface which, together with the slide surfaces of the aforementioned components, in each case forms a bearing.

### BACKGROUND OF THE INVENTION

Endoprostheses of this kind are used, for example, for replacement of the ankle joint (FR-A-2 676 917, WO-A03/075802, WO-A-2005/030098). In these, the components and the intermediate part cooperate via slide surfaces which permit flexion and extension in a sagittal plane. The sagittal plane is in this case a plane which is defined by AP direction and the vertical axis. The tibial component and the intermediate part form interacting slide surfaces which permit a rotation about the vertical axis. They can have a plane configuration in order to permit compensating movements in the AP direction and LM (lateral-medial) direction. So that the joint has degrees of freedom with respect to rotary, pivoting and/or shearing movements, which degrees of freedom correspond to the natural model, the slide surfaces correspondingly have different contours, for example a flat slide surface is combined with a slide surface which is curved in the manner of a cylindrical sleeve. Stabilization is afforded by the natural ligament apparatus.

After a prosthesis has been implanted, fibrosis often occurs, i.e. the tissue surrounding the prosthesis proliferates. As a result of unimpeded growth, the tissue can infiltrate into interstices of the prosthesis. This can cause the patient pain and/or compromise the functionality of the prosthesis.

### SUMMARY OF THE INVENTION

Starting out from the cited prior art, the object of the invention is to improve an endoprosthesis of the type mentioned in the introduction in such a way that undesired fibrosis is avoided.

The solution according to the invention lies in a prosthesis as broadly disclosed and advantageously in accordance with the detailed embodiments disclosed below.

Accordingly, in an endoprosthesis for replacement of a joint, comprising a component which is to be connected to a lower bone and which has a top slide surface, a component which is to be connected to an upper bone and which has a bottom slide surface, and an intermediate part which, on its bottom and top, has in each case a slide surface which, together with the slide surfaces of the aforementioned components, in each case define a movement plane for a bearing, the invention provides that the intermediate part is self-supporting and has a projection on at least one side face.

A number of expressions used in the context of the invention will first be explained:

The expression “movement plane” is to be understood in a broad sense and also includes curved contours.

The expression “side face” includes the surfaces of the intermediate part that are not part of the slide surfaces.

A structural part is designated as self-supporting when it withstands all the loads acting on it and when the structural part does not require any load-bearing reinforcement.

The entire load of the endoprosthesis rests on the intermediate part. The latter is therefore exposed to a considerable load along the joint axis. However, the expression “intermediate part”, within the meaning of this invention, designates only the part of the corresponding component through which force paths pass in at least one possible loaded state of the implanted prosthesis.

According to the invention, the intermediate part has a projection on at least one side face. The projection has the effect that in the event of a rotation, as also in the event of a linear movement, of the joint, and consequently also of the intermediate part, any undesired tissue material growing laterally alongside the endoprosthesis and constituting what is known as fibrosis can be forced back. Infiltration of this tissue material can be counteracted in this way or even prevented. It is thus possible to avert the danger of the joint, fitted with the endoprosthesis according to the invention, being limited in its mobility by excessive fibrosis. By virtue of the device according to the invention, it is possible to avoid the pain that usually occurs in cases of fibrosis, on account of the tissue material growing in the area of movement of the intermediate part. The configuration according to the invention means that surgical removal of this tissue material, as would perhaps otherwise be necessary, is not needed.

The projection can be of any desired shape. In principle, however, the projection should preferably have no sharp edges that could damage the tissue. Accordingly, it is preferable to round off all the edges. It is also preferable for the projection to have a convex shape.

The convexity of the projection need only be one-dimensional, such that an essentially cylindrical-sleeve shape is thus obtained; however, it can preferably also be two-dimensional, such that an essentially spherical configuration is obtained, in which case the curvature in the plane of the clamping collar and perpendicular thereto can be different.

The projection expediently extends across the entire length of the respective outer face. Although the desired effect can in principle also be achieved with a projection extending over only part of the length of an outer face, greater and therefore more favourable radii of curvature for the projection arise in the case of a convex design across the entire length. An arrangement of the projection on a medial longitudinal face of the intermediate part is particularly expedient. In the case of an implantation of the endoprosthesis according to the invention on the ankle joint for example, the medial malleolus is situated in this area. It is in this very area that undesired fibrosis may occur, the damaging results of which are prevented by virtue of the prosthesis according to the invention. The arrangement extending across the entire length also has the advantage that the desired effect of the forcing back can be achieved also in a non-rotational movement, for example a linear forward and rearward movement of the intermediate part.

The contour of the convex projection is expediently chosen such that it has the shape of an arc of a circle in plan view. Such a contour is favourable in production and gives a uniform curvature of the projection without pronounced changes to the curve profile. It is not necessary here for the centre of the circle arising from the arc to lie centrally in the intermediate part. It is expediently offset in the direction of the opposite lateral face. This results in an eccentricity, on the basis of which a stronger forcing back of the fibrotic tissue material is achieved with greater rotatory deflections of the intermediate joint part.

The outer face of the projection is preferably smooth. It can preferably be polished. This gives a form that promotes sliding, in particular under the influence of tissue fluid. The danger of tissue material tearing off or shearing off is thus effectively counteracted.

A projection can expediently also be provided on the adjacent outer faces. In the case of a rectangular design, this means that such a projection is formed on the anterior face, the posterior face and the medial face of the intermediate part.

The intermediate part is preferably made of plastic, for example polyethylene material. The projections can also be made from the same material. If this is the case, the intermediate part and the projections can also be formed as a one-piece component. The distinction between the two then arises from their respective function. The intermediate part is the part of the component that withstands the actual stress in at least one natural state of loading of the prosthesis, in other words the part through which force paths extend. The projections are the parts of the component that are free from stresses under normal circumstances and serve only to combat fibrosis.

However, the projections can also be made from a different material than the intermediate part. In this case, the projections have to be secured on the intermediate part. This can be done both mechanically and also chemically (e.g. by gluing). By means of a multi-part design of this kind, it is possible to choose the material of the projections to be as well tolerated as possible. In the case of a plastic, for example polyethylene, which can be used for the intermediate part, the tissue contact can lead to abrasion and, consequently, to possible inflammation.

To minimize this danger further and/or to increase the slidability, a protective layer can be arranged at least partially on the projections. This protective layer is preferably composed of a material that is tissue-compatible and resistant to abrasion. Depending on the configuration of the projections, the protective layer can be designed as a cap or partial collar. The protective layer itself can be secured mechanically or chemically on the projections.

### BRIEF DESCRIPTION OF THE DRAWINGS

The invention is explained below with reference to the attached drawing which depicts an advantageous illustrative embodiment of the invention and in which:

FIG. 1 shows a front view of a lower area of the shin bone together with a part of an embodiment of the endoprosthesis;

FIG. 2 shows a bottom view of the embodiment according to FIG. 1, and

FIG. 3 shows a front view of the prosthesis illustrated in FIGS. 1 and 2.

### DETAILED DESCRIPTION OF THE INVENTION

The depicted illustrative embodiment of the prosthesis according to the invention is an ankle joint prosthesis. It will be noted that the invention can also be applied to other types of endoprostheses, for example intervertebral endoprostheses. The important point is that the endoprosthesis has two bearings whose planes of movement are defined by slide surfaces.

The endoprosthesis according to the depicted illustrative embodiment basically comprises three components. The first component is a shin bone component 1 designed to be arranged on the lower end of a tibia 91. It has a plate-shaped part 10 whose bottom forms a flat slide surface 11.

The shin bone component 1 is connected fixedly to the tibia 91. The fibula 90 extends parallel to the tibia 91.

The prosthesis further comprises a component 4 which is connected to the ankle bone 92. It has a saddle-like configuration and has a convexly curved slide surface 44 on its top. It can be configured in the manner of a jacket of a cylinder. However, it can equally well have a cone-shaped design. A guide rib 46, which lies in the AP direction, is arranged on it. This serves for guiding purposes in a flexion and extension movement of the ankle joint.

An intermediate part 2 is arranged between the shin bone component 1 and the ankle bone component 4. On its top, it has a flat slide surface 21 which is configured to match the slide surface 11 of the shin bone component 1. On its bottom, the intermediate part 2 has a slide surface 24 which is congruent to the slide surface 44 of the ankle bone component 4. It additionally has a groove 26 which is designed to receive the rib 46 in a longitudinally displaceable manner. In this way, the intermediate part 2 is guided laterally in relation to the ankle bone component 4. Only flexion and extension movements are thus permitted. By contrast, the plane slide surfaces 11, 21 permit any desired movement in a horizontal plane, that is to say both longitudinal and transverse movements and also, in particular, a rotation about the vertical axis.

The shin bone component 1 and the ankle bone component 4 are expediently made of metal, for example a cobalt-chromium alloy provided on its respective outer face with a coating that promotes bone growth (for example calcium phosphate). The intermediate part 2, by contrast, is preferably made of a plastic material that promotes sliding, in particular polyethylene. However, this is not intended to rule out the possibility of also using other materials with sufficient strength and slidability.

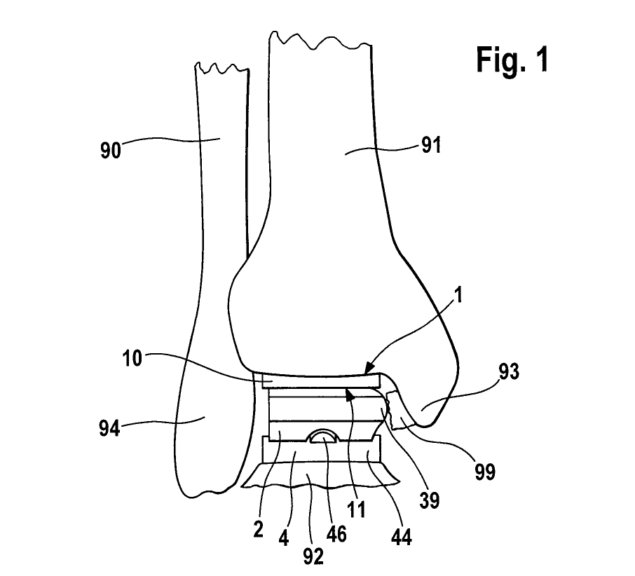

FIG. 1 shows the endoprosthesis at its intended site of implantation at the distal end of the tibia 91. At its distal end, the tibia 91 forms a plateau on which the shin bone component 1 of the endoprosthesis according to the invention is arranged. This plateau is limited in the medial direction by a continuation of the tibia 91, the so-called medial malleolus 93, and in the lateral direction by a corresponding continuation of the fibula 90, namely the lateral malleolus 94. They enclose the plateau of the tibia 91, and therefore the shin bone component 1 of the endoprosthesis, like a fork. This can be seen clearly in FIG. 2.

It has been shown that, some time after implantation, a formation of tissue material (fibrosis) 99 often occurs in the area between the medial malleolus 93 and the intermediate part. This can cause pain which not only could be very unpleasant for the patient but in quite a few cases could also necessitate surgical intervention to remove the tissue material 99. According to the invention, in order to avoid or reduce the fibrotic tissue material 99, a projection 39 is formed at least on a longitudinal face 33 of the intermediate part 2, expediently on the medial face. The projection extends outward relative to a contour which is congruent with the intermediate part 2. The projection preferably has an arc-shaped outer contour, the arc extending across the entire length. The projection 39 is expediently curved in two dimensions, that is to say it has a spherical surface shape. The radii of curvature here are of different sizes, a weak curvature in the horizontal plane (as is shown in FIG. 2) and a stronger curvature in a frontal plane (as is shown in FIG. 1). To obtain the largest possible radius of curvature in the horizontal plane, the midpoint 30 of the circle defined by the radius of curvature preferably does not lie centrally in the intermediate part 2 but is instead eccentrically offset in the lateral direction and preferably also in the frontal direction. The outer face of the projection 39 is smooth.

The illustrative embodiment shown represents one option, specifically one in which the front face and the rear face of the intermediate part 2 are also each provided with a projection 39′ and 39″, respectively. As can be seen from FIG. 3, they deviate in shape from the projection 39, namely being of cylindrical-sleeve shape. In addition, they do not extend over the entire height of the intermediate part 2. A transition of equal curvature between the projections 39, 39′, 39″ is not necessary, but the geometries are expediently chosen such that the transition is stepless. The lateral longitudinal face of the intermediate part 2 expediently has no projection. This serves to ensure free movement of the intermediate part 2. This also has the advantage of providing an unambiguous orientation of the intermediate part 2, as a result of which the danger of its being fitted in an incorrect position is reduced.

The projection 39 is normally designed in one piece with the intermediate part 2. However, this should not rule out the possibility of choosing a multi-part construction in which the projection 39 is designed as a separate part and is secured on the intermediate part 2 by suitable securing means. The latter option affords the advantage that, for the projection 39, it is possible to choose a material which especially promotes sliding and is especially suitable for contact with the fibrotic tissue material 99, without compromising the mechanical load-bearing capacity of the intermediate part 2.

In the areas where contact with tissue material 99 is to be expected, the projection 39 is provided with a protective layer 50. By this means, there is no danger of damage to the surrounding tissue 99 by abrasion or the like. During movement of the endoprosthesis, particularly during rotation, but also during a forward or rearward movement in the longitudinal direction, the projection 39 ensures that fibrotic tissue material 99 is forced back. This effectively counteracts infiltration of the fibrotic tissue 99 into the area of the endoprosthesis.

Finally, it will be noted that the configuration according to the invention of an intermediate part 2 with a projection 39 is not limited to ankle-joint endoprostheses.

1. An endoprosthesis for replacement of a joint, comprising a component configured to be connected to an upper bone and which has a bottom slide surface, a component configured to be connected to a lower bone and which has a top slide surface, and an intermediate part which, on its bottom and top, has in each case a slide surface which, together with the slide surfaces of the components, in each case define a movement plane for a bearing, wherein the intermediate part is self-supporting and has a projection on at least one side face.

2. The endoprosthesis according to claim 1, wherein the projection is a convex bulge.

3. The endoprosthesis according to claim 1 or 2, wherein the projection extends across the entire length of the respective outer face.

4. The endoprosthesis according to claim 1 or 2, wherein the projection is formed on a medial longitudinal face of the intermediate part.

5. The endoprosthesis according to claim 1 or 2, wherein the projection has an arc-shaped contour in plan view.

6. The endoprosthesis according to claim 5, wherein the arc-shaped contour follows an arc of a circle whose centre is offset towards the opposite side of the intermediate part.

7. The endoprosthesis according to claim 1 or 2, wherein additional projections are formed on an anterior face and a posterior face of the intermediate part.

8. The endoprosthesis according claim 1 or 2, wherein the intermediate part is made of plastic.

9. The endoprosthesis according to claim 1 or 2, wherein the projections are formed in one piece with the intermediate part.

10. The endoprosthesis according to claim 1 or 2, wherein the projections are formed separately from the intermediate part and are secured thereon.

11. The endoprosthesis according to claim 1 or 2, wherein the projections are covered with a protective layer.

12. The endoprosthesis according to claim 11, wherein the protective layer is designed as a cap or partial collar.

13. The endoprosthesis according to claim 11, wherein the protective layer is secured mechanically on the projections.

14. The endoprosthesis according to claim 11, wherein the protective layer is secured chemically on the projections.

15. The endoprosthesis according to claim 1, wherein the slide surfaces of the intermediate part have different contours.
